(19) United States
(12) Patent Application Publication
(10) Pub. No.: US 2014/0213890 A1
(43) Pub. Date: Jul. 31, 2014

(54) METHOD AND APPARATUS FOR GATHERING BODILY FLUID DYNAMIC PRESSURE MEASUREMENTS

(71) Applicant: PACESETTER, INC., (US)

(72) Inventors: Hedi Razavi, San Jose, CA (US); Timothy A. Fayram, Gilroy, CA (US)

(73) Assignee: PACESETTER, INC., Sunnyvale, CA (US)

(21) Appl. No.: 13/756,386

(22) Filed: Jan. 31, 2013

(51) Int. Cl. A61B 5/0215; A61B 5/06

(52) U.S. Cl. CPC A61B 5/0215; A61B 5/061
USPC 600/424; 600/486

(57) ABSTRACT

An apparatus and method for gathering bodily fluid dynamic pressure measurements including placing a delivery tool in a region of interest (ROI), wherein the delivery tool includes a sensor, wherein the sensor is positioned in a substantially perpendicular direction to a flow direction of the ROI; measuring a sensor displacement for a time period; and determining a pressure measurement in the ROI using the sensor displacement.

### FIELD

This disclosure relates generally to techniques for gathering pressure measurements. More particularly, the disclosure relates to gathering bodily fluid dynamic pressure measurements.

### BACKGROUND

Gathering pressure measurements in a body cavity is one of the tools of medical diagnosis and treatment. For example, intravascular and intracardiac hemodynamic measurements such as blood pressure measurements are performed frequently in catheterization suites to evaluate disease state severity as in the case of pulmonary hypertension, to guide therapy decisions as in the case of Fractional Flow Reserve (FFR) measurements to evaluate the degree of vascular stenosis and decide on the appropriate therapy, and/or to predict response to therapy and survival as in the case of cardiac resynchronization therapy in patients with heart failure.

Currently, intravascular and intracardiac pressure measurements are performed using either pressure wires which can suffer from measurement drifts and increased radiation exposure, or using non-invasive Doppler ultrasound which suffers from inaccuracies. A need exists to reduce radiation exposure, for example, by visualizing real-time location of tools on a short pre-recorded fluoroscopy image of the anatomy of interest. A desire also exists to improve accuracy in intravascular or intracardiac pressure measurements without additional radiation exposure.

### SUMMARY

According to one aspect, a method for gathering bodily fluid dynamic pressure measurements including placing a delivery tool in a region of interest (ROI), wherein the delivery tool comprises a sensor, wherein the sensor is positioned in a substantially perpendicular direction to a flow direction of the ROI; measuring a sensor displacement for a time period; and determining a pressure measurement in the ROI using the sensor displacement.

According to another aspect, a method for gathering bodily fluid dynamic pressure measurements including measuring a sensor displacement of an electromagnetic sensor positioned in a region of interest (ROI) for a time period, wherein the electromagnetic sensor is positioned in a substantially perpendicular direction to a flow direction of the ROI; and determining a pressure measurement in the ROI using the sensor displacement by: a) calculating a time-dependent acceleration, a, of the sensor according to x=x₀+v₀t+½ at², where t is the time period, x is the sensor displacement, x₀ is the initial sensor position at the beginning of the time period t, v₀ is the initial sensor velocity at the beginning of the time period t; b) calculating a force F exerted on the sensor by a flow flowing in the flow direction according to F=ma, where a is the time-dependent acceleration of the sensor and m is the mass of the sensor; and c) calculating a pressure P exerted on the sensor according to the equation P=F/A, where F is force exerted on the sensor and A is the cross-sectional dimension of the sensor that is perpendicular to the flow direction.

According to yet another aspect, a device for gathering bodily fluid dynamic pressure measurements comprising a processor and a memory, the memory containing program code executable by the processor for performing the following: placing a delivery tool in a region of interest (ROI), wherein the delivery tool comprises a sensor, wherein the sensor is positioned in a substantially perpendicular direction to a flow direction of the ROI; measuring a sensor displacement for a time period; and determining a pressure measurement in the ROI using the sensor displacement.

Advantages of the present disclosure may include reducing radiation exposure, for example, by reducing the need for fluoroscopy, and reducing calibration drifts and/or measurement errors.

It is understood that other aspects will become readily apparent to those skilled in the art from the following detailed description, wherein it is shown and described various aspects by way of illustration. The drawings and detailed description are to be regarded as illustrative in nature and not as restrictive.

### BRIEF DESCRIPTION OF THE DRAWINGS

FIG. 1 illustrates an example of a sensor associated with a delivery tool within a blood vessel wall.

FIG. 2 illustrates an example of a graph indicating sensor position over time.

FIG. 3 illustrates an example of a flow diagram for gathering bodily fluid dynamic pressure measurements.

FIG. 4 illustrates an example of a device comprising a processor in communication with a memory for executing the processes of gathering bodily fluid dynamic pressure measurements.

### DETAILED DESCRIPTION

The detailed description set forth below in connection with the appended drawings is intended as a description of various aspects of the present disclosure and is not intended to represent the only aspects in which the present disclosure may be practiced. Each aspect described in this disclosure is provided merely as an example or illustration of the present disclosure, and should not necessarily be construed as preferred or advantageous over other aspects. The detailed description includes specific details for the purpose of providing a thorough understanding of the present disclosure. However, it will be apparent to those skilled in the art that the present disclosure may be practiced without these specific details. In some instances, well-known structures and devices are shown in block diagram form in order to avoid obscuring the concepts of the present disclosure. Acronyms and other descriptive terminology may be used merely for convenience and clarity and are not intended to limit the scope of the present disclosure.

In one aspect, the present disclosure relates to providing accurate positional information of a sensor located within the body of a patient. In one example, the sensor measures intravascular or intracardiac blood pressure. For example, the sensor is embedded, attached to or part of a delivery tool such as a catheter, a guidewire, a stylet or a lead. In one aspect, the delivery tool allows the positioning of the sensor such that it is positioned in a perpendicular direction to the direction of fluid flow. In one example, the fluid flow is blood flow. In another example, the fluid flow is spinal fluid flow or other bodily fluid flow. In yet another example, the fluid flow is air flow within the lungs. One skilled in the art would understand that the examples of fluid flow listed herein are just examples and that other types of fluid or air flow within a body cavity (e.g., a human body cavity), although not specifically mentioned herein, are part of the scope and spirit of the present disclosure.

In one example, with each heartbeat, the sensor moves according to the forces applied to it by the fluid flow (e.g., blood flow). The acceleration of the sensor is indicative of the pressures applied by the forces of the fluid flow. FIG. 1 illustrates an example of a sensor associated with a delivery tool within a blood vessel lumen. In FIG. 1, the delivery tool is shown in the shape of an “L” where it includes a tubular body and a tip. In one example, the tubular body may be hollow or it may be solid. The tubular body makes up the longer portion of the “L” shape while the tip makes up the shorter portion of the “L” shape. As shown in FIG. 1, the sensor is positioned at the tip of the delivery tool. In one example, the sensor is positioned in a perpendicular direction to the direction of blood flow. This positioning of the sensor allows the sensor to be deflected by the force of the blood flow as indicated by “X” shown in FIG. 1. “X” indicates the amount of deflection of the sensor, which may be caused in part by the force of the blood flow.

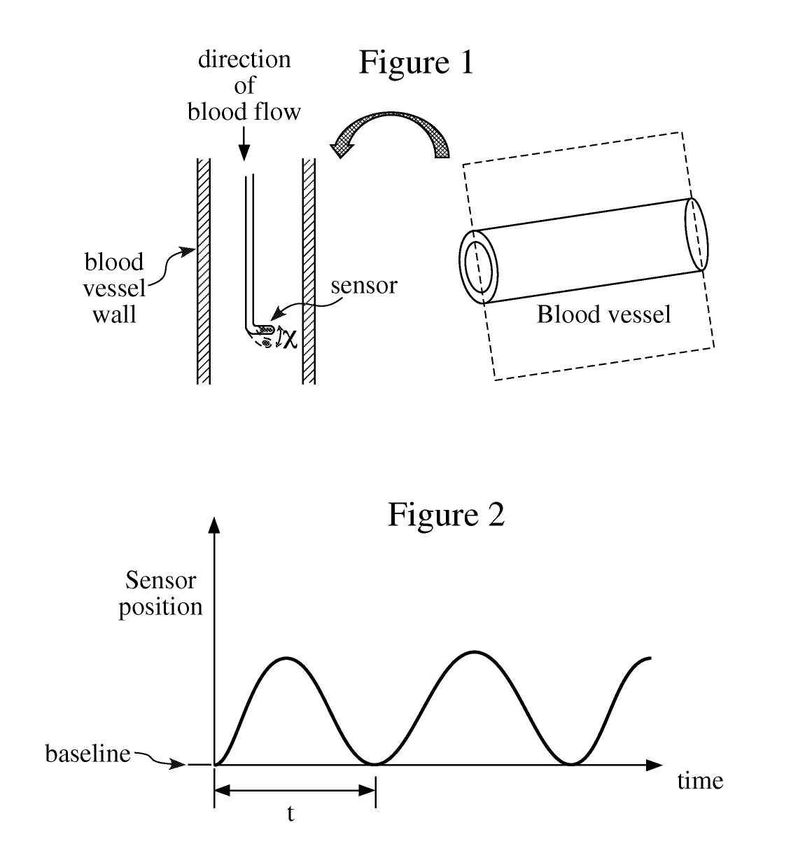

FIG. 2 illustrates an example of a graph indicating sensor position over time. “t” indicates the time period of the graph. As shown in FIG. 2, the sensor position starts at a baseline, then increases to a maximum and then decreases back to the baseline in the time period t. In this example, the baseline indicates the sensor's starting position. In one example, the time period t corresponds to a cardiac cycle. In another example, the time period t is a fraction of a cardiac cycle. In yet another example, the time period t is greater than one cardiac cycle.

In one aspect, given the position information measured by the sensor, the acceleration of the sensor may be calculated according to x=x₀+v₀t+½ at² or any higher dimensional order or numerical variations where t is a time period, x is the sensor displacement, x₀ is the initial sensor position at the beginning of the time period t, and v₀ is the initial sensor velocity at the beginning of the time period t. In one example, v₀ is zero because initially the sensor is not moving. Once the acceleration, a, is calculated and given the mass of the sensor m, Newton's second law F=ma and any higher order or numerical variations of it may be used to derive the total force F applied to the sensor. Given the perpendicular cross-section A of the sensor, pressure P applied to the sensor may be calculated according to P=F/A or any other higher order or numerical variations. Although specific equations are presented herein as examples, one skilled in the art would understand that other equations for calculating pressure, including more complex versions of the equations presented herein (e.g., higher dimensions, higher orders or numerical formats, etc.) are within the scope and spirit of the present disclosure.

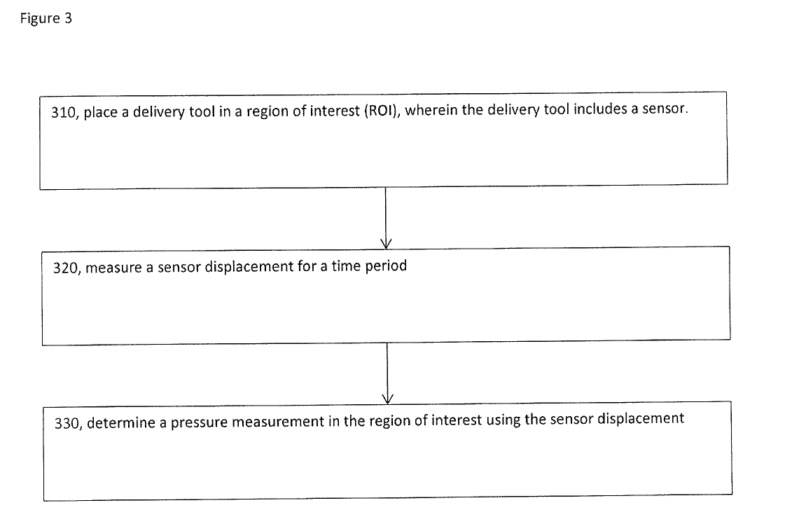

FIG. 3 illustrates an example of a flow diagram for gathering bodily fluid dynamic pressure measurements. In block 310, place a delivery tool in a region of interest (ROI), wherein the delivery tool includes a sensor. In one example, the sensor is positioned in a substantially perpendicular direction to a flow direction of the region of interest (ROI). In one example, a fluoroscopy image (e.g., a pre-recorded fluoroscopy image) of the region of interest is used for placing the delivery tool. In one example, the fluoroscopy image is a 2-dimensional image, while in another example, the fluoroscopy image is a live 3-dimensional image. In another example, one or more of a magnetic resonance imaging (MRI) image, an ultrasonic image, or a computed tomography scan (CT scan) or computed axial tomography scan (CAT scan) image of the region of interest is used for placing the delivery tool.

In one example, the delivery tool is a catheter, a guidewire, a stylet or a lead. One skilled in the art would understand that the list of examples of delivery tools is not exclusive and that other delivery tools not listed herein may be used within the scope and spirit of the present disclosure.

In one example, the delivery tool has an “L” shape in one configuration. In this “L” shape, the delivery tool includes a tubular body and a tip where the tubular body makes up the longer portion of the “L” shape while the tip makes up the shorter portion of the “L” shape. The delivery tool includes an inserting configuration and a launched configuration. In the inserting configuration, the tip is a linear extension of the tubular body. In the launched configuration, the tip forms a substantially “L” shape with the tubular body. The tubular body may be hollow or it may be solid. The delivery tool may include a hinging component for configuring the delivery tool from the inserting configuration to the launched configuration. In one example, the hinging component is a hinge that couples the tubular body and the tip. In another example, one or more wire components are rotated to pivot the tip to form the L shape. The wire component(s) may be situated within the hollow tubular body of the delivery tool. In yet another example, the delivery tool includes shaped memory alloy such that the shape of the delivery tool is changed (for example, from an initial configuration to a launched configuration) when a particular temperature is reached, or when the delivery tool is in contact with a particular environment or experiences an environmental variable.

In one example, the sensor is housed at or near the tip of the delivery tool. The delivery tool may include a loaded spring for repositioning the sensor to an initial position following the sensor displacement from pressure of the fluid flow within the region of interest. The sensor may be an electromagnetic sensor or an ultrasonic sensor. However, one skilled in the art would understand that other types of sensors used for measuring displacement are also within the scope and spirit of the present disclosure.

In block 320, measure a sensor displacement for a time period. In block 330, determine a pressure measurement in the region of interest using the sensor displacement. In one aspect, the pressure measurement is determined by calculating a time-dependent acceleration of the sensor, calculating a force exerted on the sensor based on the time-dependent acceleration and calculating a pressure exerted on the sensor based on the force.

In another aspect, the pressure measurement is determined by performing the following: First, calculate a time-dependent acceleration, a, of the sensor according to the equation x=x₀+v₀t+½ at² or any other higher order or numerical variations where t is the time period, x is the sensor displacement, x₀ is the initial sensor position at the beginning of the time period t, and v₀ is the initial sensor velocity at the beginning of the time period t. In one example, v₀ is zero because initially the sensor is not moving. Second, calculate a force F exerted on the sensor by a flow flowing in the flow direction according to the equation F=ma or any other higher order or numerical variations, where a is the time-dependent acceleration of the sensor and m is the mass of the sensor. Third, calculate a pressure P exerted on the sensor according to the equation P=F/A or any other higher order or numerical variations, where F is force exerted on the sensor and A is the cross-sectional dimension of the sensor that is perpendicular to the flow direction. In one example, the mass and area of the tip of the delivery tool where the sensor is positioned are negligible compared to the mass and area of the sensor. In another example, the mass and area values used in the calculation may take the mass and area of the tip into account if they are not deemed to be negligible. In another example where the delivery tool includes a loaded spring at or near the tip, the mass of the loaded spring may be included as part of the sensor mass in the calculation. One skilled in the art would understand that the value of the mass m in the calculation may include any other mass value associated with the delivery tool that is needed to be included so to provide an accurate measurement based on the equations described herein.

In yet another aspect, the pressure measurement is determined by performing the following: First, calculate a force F exerted on the sensor by a flow in the flow direction according to the equation F=kx or any other higher order or numerical variations, where k is an effective spring constant of the sensor and x is the sensor displacement. Second, calculate a pressure P exerted on the sensor according to the equation P=F/A or any other higher order or numerical variations, where F is the force exerted on the sensor and A is the cross-sectional dimension of the sensor that is perpendicular to the flow direction.

In one example, the value of the effective spring constant of the sensor k is unknown, however, the ratio of k/m is known where m is the mass of the sensor. However, one skilled in the art would understand that if the mass of the tip of the delivery tool and/or the mass of a loaded spring at or near the tip are not negligible, the value of m as used in the calculation may include the mass of the tip and/or the mass of the loaded spring. And, one skilled in the art would also understand that the value of the mass m in the calculation may include any other mass value associated with the delivery tool that is needed to be included so as to provide an accurate measurement based on the equations described herein.

In this example, the pressure measurement is determined by performing the following: First, calculate a time-dependent acceleration, a, of the sensor according to the equation a=(k/m)*x or any higher order or numerical variations, where k is a spring constant of the sensor, m is the mass of the sensor and x is the sensor displacement. Second, calculate a force F exerted on the sensor by a flow flowing in the flow direction according to the equation F=ma or any higher order or numerical variations, where m is the mass of the sensor and a is the time-dependent acceleration of the sensor. Third, calculate a pressure P exerted on the sensor according to the equation P=F/A or any higher order or numerical variations, where F is force exerted on the sensor and A is the cross-sectional dimension of the sensor that is perpendicular to the flow direction.

In one example, the steps illustrated in FIG. 3 are repeated for multiple time periods. And, the pressure measurements are averaged over the time periods to obtain one average pressure measurement. In one example, the pressure measurements are averaged over the number of cardiac cycles.

In one aspect, the region of interest (ROI) is an artery or a vein, for example, a coronary artery or a cardiac vein. In one example, the region of interest (ROI) is a cardiac chamber, for example, in the ventricles or atria. In another example, the ROI is a cerebral spinal fluid cavity. In another example, the ROI is the trachea, the bronchia or the lung cavity. One skilled in the art would understand that the list of examples of ROI is not exclusive and that the present disclosure may be equally applicable to other bodily cavities. In one example, the time period t is related to a cardiac cycle. However, depending on the ROI, the time period t may be related, for example, to a respiratory cycle or another bodily cycle.

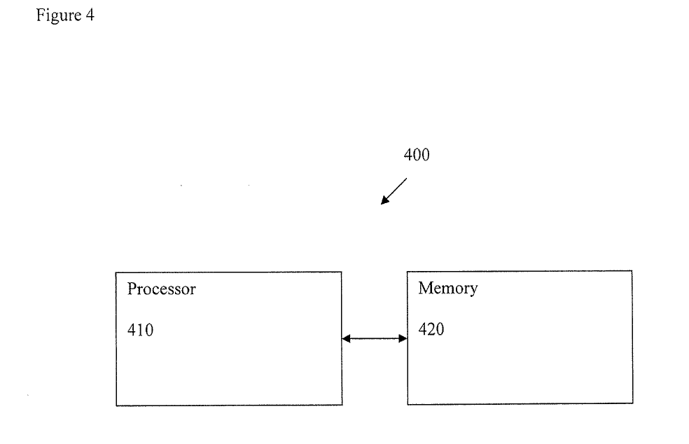

FIG. 4 illustrates an example of a device 400 comprising a processor 410 in communication with a memory 420 for executing the processes of gathering bodily fluid dynamic pressure measurements. In one example, the device 400 is used to implement the algorithm illustrated in FIG. 3. In one aspect, the memory 420 is located within the processor 410. In another aspect, the memory 420 is external to the processor 410. In one aspect, the processor includes circuitry for implementing or performing the various flow diagrams, logical blocks and/or modules described herein.

While for purposes of simplicity of explanation, the methodologies are shown and described as a series of acts, it is to be understood and appreciated that the methodologies are not limited by the order of acts, as some acts may, in accordance with one or more aspects, occur in different orders and/or concurrently with other acts from that shown and described herein. For example, those skilled in the art will understand and appreciate that a methodology could alternatively be represented as a series of interrelated states or events. Moreover, not all illustrated acts may be required to implement a methodology in accordance with one or more aspects.

The previous description of the disclosed aspects is provided to enable any person skilled in the art to make or use the present disclosure. Various modifications to these aspects will be readily apparent to those skilled in the art, and the generic principles defined herein may be applied to other aspects without departing from the spirit or scope of the disclosure.

1. A method for gathering bodily fluid dynamic pressure measurements comprising:
placing a delivery tool in a region of interest (ROI), wherein the delivery tool comprises a sensor, wherein the sensor is positioned in a substantially perpendicular direction to a flow direction of the ROI;
measuring a sensor displacement for a time period; and
determining a pressure measurement in the ROI using the sensor displacement.

2. The method of claim 1, further comprising determining the pressure measurement by calculating a time-dependent acceleration of the sensor, calculating a force exerted on the sensor based on the time-dependent acceleration, and calculating a pressure exerted on the sensor based on the force.

3. The method of claim 1, further comprising
calculating a time-dependent acceleration a of the sensor according to x=x₀+v₀t+½ at², where t is the time period, x is the sensor displacement, x₀ is the initial sensor position at the beginning of the time period t, and v₀ is the initial sensor velocity at the beginning of the time period t;
calculating a force F exerted on the sensor by a flow flowing in the flow direction according to F=ma, where a is the time-dependent acceleration of the sensor and m is the mass of the sensor; and
calculating a pressure P exerted on the sensor according to the equation P=F/A, where F is force exerted on the sensor and A is the cross-sectional dimension of the sensor that is perpendicular to the flow direction.

4. The method of claim 1, further comprising
calculating a force F exerted on the sensor by a flow flowing in the flow direction according to F=kx, where k is an effective spring constant of the sensor and x is the sensor displacement; and
calculating a pressure P exerted on the sensor according to P=F/A, where F is force exerted on the sensor and A is the cross-sectional dimension of the sensor that is perpendicular to the flow direction.

5. The method of claim 1, further comprising
calculating a time-dependent acceleration a of the sensor according to a=(k/m)*x, where k is a spring constant of the sensor, m is the mass of the sensor and x is the sensor displacement;
calculating a force F exerted on the sensor by a flow flowing in the flow direction according to the equation F=ma, where m is the mass of the sensor and a is the time-dependent acceleration of the sensor; and
calculating a pressure P exerted on the sensor according to the equation P=F/A, where F is force exerted on the sensor and A is the cross-sectional dimension of the sensor that is perpendicular to the flow direction.

6. The method of claim 1 further comprising using an image of the region of interest (ROI) for placing the delivery tool.

7. The method of claim 6, wherein the image is one of the following: a fluoroscopy image; a magnetic resonance imaging (MRI) image, an ultrasonic image, a computed tomography scan (CT scan) or a computed axial tomography scan (CAT scan) image.

8. The method of claim 1, wherein the delivery tool comprises a tubular body and a tip, and wherein the sensor is housed on the tip.

9. The method of claim 8, wherein the delivery tool includes an inserting configuration where the tip is a linear extension of the tubular body, and a launched configuration where the tip forms a substantially “L” shape with the tubular body.

10. The method of claim 1, wherein the delivery tool comprises a loaded spring for repositioning the sensor to an initial position following occurrence of the sensor displacement.

11. The method of claim 1, wherein the sensor is an electromagnetic sensor or an ultrasonic sensor.

12. The method of claim 11, wherein the region of interest (ROI) is a coronary artery or a cardiac vein.

13. The method of claim 12, wherein the delivery tool is one of the following: a catheter, a guidewire, a stylet or a lead.

14. The method of claim 13, wherein the time period is a cardiac cycle.

15. The method of claim 1, further comprising
measuring at least two sensor displacements for at least two time periods;
determining at least two pressure measurements in the ROI using the at least two sensor displacements; and
averaging the at least two pressure measurements over the at least two time periods.

16. A method for gathering bodily fluid dynamic pressure measurements comprising:
measuring a sensor displacement of an electromagnetic sensor positioned in a region of interest (ROI) for a time period, wherein the electromagnetic sensor is positioned in a substantially perpendicular direction to a flow direction of the ROI; and
determining a pressure measurement in the ROI using the sensor displacement by:
a) calculating a time-dependent acceleration a of the sensor according to x=x₀+v₀t+½ at², where t is the time period, x is the sensor displacement, x₀ is the initial sensor position at the beginning of the time period t, v₀ is the initial sensor velocity at the beginning of the time period t;
b) calculating a force F exerted on the sensor by a flow flowing in the flow direction according to F=ma, where a is the time-dependent acceleration of the sensor and m is the mass of the sensor; and
c) calculating a pressure P exerted on the sensor according to the equation P=F/A, where F is force exerted on the sensor and A is the cross-sectional dimension of the sensor that is perpendicular to the flow direction.

17. A device for gathering bodily fluid dynamic pressure measurements comprising a processor and a memory, the memory containing program code executable by the processor for performing the following:
placing a delivery tool in a region of interest (ROI), wherein the delivery tool comprises a sensor, wherein the sensor is positioned in a substantially perpendicular direction to a flow direction of the ROI;
measuring a sensor displacement for a time period; and
determining a pressure measurement in the ROI using the sensor displacement.

18. The device of claim 17, wherein the memory further comprising program code for determining the pressure measurement by calculating a time-dependent acceleration of the sensor, calculating a force exerted on the sensor based on the time-dependent acceleration, and calculating a pressure exerted on the sensor based on the force.

19. The device of claim 17, wherein the memory further comprising program code for:
calculating a time-dependent acceleration a of the sensor according to x=x₀+v₀t+½ at², where t is the time period, x is the sensor displacement, x₀ is the initial sensor position at the beginning of the time period t, and v₀ is the initial sensor velocity at the beginning of the time period t;
calculating a force F exerted on the sensor by a flow flowing in the flow direction according to F=ma, where a is the time-dependent acceleration of the sensor and m is the mass of the sensor; and
calculating a pressure P exerted on the sensor according to the equation P=F/A, where F is force exerted on the sensor and A is the cross-sectional dimension of the sensor that is perpendicular to the flow direction.

20. The device of claim 17, wherein the memory further comprising program code for:
calculating a force F exerted on the sensor by a flow flowing in the flow direction according to F=kx, where k is an effective spring constant of the sensor and x is the sensor displacement; and
calculating a pressure P exerted on the sensor according to P=F/A, where F is force exerted on the sensor and A is the cross-sectional dimension of the sensor that is perpendicular to the flow direction.

21. The device of claim 17, wherein the memory further comprising program code for:
calculating a time-dependent acceleration a of the sensor according to a=(k/m)*x, where k is a spring constant of the sensor, m is the mass of the sensor and x is the sensor displacement;
calculating a force F exerted on the sensor by a flow flowing in the flow direction according to the equation F=ma, where m is the mass of the sensor and a is the time-dependent acceleration of the sensor; and
calculating a pressure P exerted on the sensor according to the equation P=F/A, where F is force exerted on the sensor and A is the cross-sectional dimension of the sensor that is perpendicular to the flow direction.

22. The device of claim 17, wherein the memory further comprising program code for using an image of the region of interest (ROI) for placing the delivery tool.

23. The device of claim 22, wherein the image is one of the following: a fluoroscopy image; a magnetic resonance imaging (MRI) image, an ultrasonic image, a computed tomography scan (CT scan) or a computed axial tomography scan (CAT scan) image.

24. The device of claim 17, wherein the delivery tool comprises a tubular body and a tip, and wherein the sensor is housed on the tip.

25. The device of claim 24, wherein the delivery tool includes an inserting configuration where the tip is a linear extension of the tubular body, and a launched configuration where the tip forms a substantially “L” shape with the tubular body.

26. The device of claim 25, wherein the region of interest (ROI) is a coronary artery or a cardiac vein and the time period is a cardiac cycle.

27. The device of claim 26, wherein the sensor is an electromagnetic sensor or an ultrasonic sensor.

28. The device of claim 27, wherein the delivery tool comprises a loaded spring for repositioning the sensor to an initial position following occurrence of the sensor displacement.
